(19) United States
(12) Patent Application Publication
(10) Pub. No.: US 2022/0119767 A1
(43) Pub. Date: Apr. 21, 2022

(54) NATURAL KILLER CELL INDUCED CELLULAR VESICLES FOR CANCER THERAPY

(71) Applicant: The Regents of the University of California, Oakland, CA (US)

(72) Inventors: Young Jik Kwon, Irvine, CA (US); Melissa Thone, Irvine, CA (US); Dominique Antoinette Ingato, Irvine, CA (US)

(21) Appl. No.: 17/433,573

(22) PCT Filed: Feb. 28, 2020
(86) PCT No.: PCT/US2020/020539

§ 371 Date: Aug. 24, 2021

Related U.S. Application Data
(60) Provisional application No. 62/812,908, filed on Mar. 1, 2019.

(51) Int. Cl. C12N 5/0783; A61K 35/17; A61P 35/00

(52) U.S. Cl. CPC C12N 5/0646; A61P 35/00; A61K 35/17

(57) ABSTRACT

The disclosure provides methods for the production of induced cellular vesicles from natural killer cells and uses thereof, including as a cancer therapy.

### CROSS REFERENCE TO RELATED APPLICATIONS

This application claims priority under 35 U.S.C. § 119 from Provisional Application Ser. No. 62/812,908 filed Mar. 1, 2019, the disclosure of which is incorporated herein by reference.

### STATEMENT OF GOVERNMENT SUPPORT

This invention was made with Government support under Grant No. DGE-1321846, awarded by the National Science Foundation. The Government has certain rights in the invention.

### TECHNICAL FIELD

The disclosure provides methods for the production of induced cellular vesicles from natural killer cells and uses thereof, including as a treatment option for subjects with cancer.

### BACKGROUND

As cancer cells progress into tumors, a patient's own immune system can recognize and destroy these abnormal cells using natural killer (NK) cells. Upon recognition of abnormal cells, NK cells form an immunological synapse and release cytotoxic granules to the target cell, inducing cell death. NK cell cytotoxicity is also regulated by recognition of self-associated molecules (such as major histocompatibility complex or MHC class I), which inhibit cytotoxic granule release to non-target cells. In this way, NK cells can act as targeted and effective therapeutics for cancer.

### SUMMARY

The disclosure provides for methods that efficiently produce micrometer and nanometer sized induced cellular vesicles from natural killer cells by using chemical agents (e.g., paraformaldehyde, dithiothreitol, and N-ethylmaleimide) to rapidly induce vesicle formation by the natural killer cells. The methods of the disclosure are significantly faster, more efficient, and produce higher yields compared to current vesicle production techniques. Moreover, the methods disclosed herein have the added benefit of locking the presentation state of the natural killer cells, insuring control and homogeneity. Accordingly, the methods of the disclosure facilitate the use of natural killer cell vesicle-based therapeutics for treating diseases and conditions, such as cancer. The natural killer cell induced cellular vesicles disclosed herein are more controllable and therefore less risky than designing new whole cell natural killer cell therapies. The natural killer cell induced cellular vesicles of the disclosure can be loaded with various therapeutics, including anticancer therapeutics, thereby augmenting the therapeutic effectiveness of the induced cellular vesicles for treating diseases, like cancer. Further, natural killer induced cellular vesicles exhibit natural targeting ability and reduce toxic side effects to non-cancerous cells.

In a particular embodiment, the disclosure provides a method to produce natural killer cell induced cellular vesicles (NK ICVs), comprising: contacting natural killer cells with one or more sulfhydryl blocking agents to blebbing of natural killer cells to produce NK ICVs; optionally, isolating or purifying the NK ICVs. In further embodiment, the natural killer cells are human natural killer cells. In yet a further embodiment of any of the foregoing embodiments, the human natural killer cells are immortalized human natural killer cells. In yet a further embodiment of any of the foregoing embodiments, the immortalized human natural killer cells are selected from NK-92, NK-92MI, NKL, KYHG-1, and NKG. In yet a further embodiment of any of the foregoing embodiments, the immortalized human natural killer cells are either NK-92 cells or NK-92MI cells. In yet a further embodiment of any of the foregoing embodiments, the human natural killer cells are differentiated from human embryonic stem cells (hESCs) or induced pluripotent stem cells (iPSCs) from a human subject. In yet a further embodiment of any of the foregoing embodiments, the iPSCs are T-cell peripheral blood cell (PBC)-derived iPSCs. In yet a further embodiment of any of the foregoing embodiments, the T-cell PBC-derived iPSCs are differentiated to NK cells by: culturing PBC-derived iPSCs with OP9 cells to form CD34+ differentiated cells; co-culturing CD34+ differentiated cells with OP9-DLL1 cells to form CD45+CD56+ natural killer cells. In yet a further embodiment of any of the foregoing embodiments, the human natural killer cells are isolated from peripheral blood mononuclear cells or washed leukapheresis samples of one or more human subjects. In yet a further embodiment of any of the foregoing embodiments, the human natural killer cells are isolated from peripheral blood mononuclear cells or washed leukapheresis samples using immunomagnetic negative selection, whereby non-natural killer cells are labeled with antibodies and magnetic particles and then removed with a magnet, leaving natural killer cells. In yet a further embodiment of any of the foregoing embodiments, the human natural killer cells have been genetically modified to express transgenes encoding antigen(s) and/or receptor(s). In yet a further embodiment of any of the foregoing embodiments, where the human natural killer cells were genetically modified by use a viral vector system. In yet a further embodiment of any of the foregoing embodiments, the viral vector system is a lentiviral, or a retroviral vector system. In yet a further embodiment of any of the foregoing embodiments, the human natural killer cells have been genetically modified to express a chimeric antigen receptor (CAR), and wherein the NK ICVs produced are CAR-NK ICVs. In yet a further embodiment of any of the foregoing embodiments, the natural killer cells are contacted with the one or more sulfhydryl blocking agents for 3 min to 24 h. In yet a further embodiment of any of the foregoing embodiments, the one or more sulfhydryl blocking agents are selected from the group consisting of mercury chloride, p-chloromercuribenzene sulfonic acid, auric chloride, p-chloromercuribenzoate, chlormerodrin, meralluride sodium, iodoacetmide, paraformaldehyde, dithiothreitol, and N-ethylmaleimide. In yet a further embodiment of any of the foregoing embodiments, the one or more sulfhydryl blocking agents are paraformaldehyde, or paraformaldehyde and dithiothreitol. In yet a further embodiment of any of the foregoing embodiments, paraformaldehyde is used at a concentration from 20 mM to 250 mM. In yet a further embodiment of any of the foregoing embodiments, dithiothreitol is used at a concentration of 1 mM to 4 mM. In yet a further embodiment of any of the foregoing embodiments, the one or more sulfhydryl blocking agents is N-ethylmaleimide. In yet a further embodiment of any of the foregoing embodiments, N-ethylmaleimide is used at a concentration of 1 mM to 20 mM. In yet a further embodiment of any of the foregoing embodiments, wherein micrometer sized NK ICVs are isolated or purified. In yet a further embodiment of any of the foregoing embodiments, nanometer sized NK ICVs are isolated or purified.

In a certain embodiment, the disclosure provides for natural killer cell induced cellular vesicles (NK ICVs) produced by a method disclosed herein. In a further embodiment, the NK ICVs are loaded with one or more small molecule therapeutic compounds or agents. In yet a further embodiment of any of the foregoing embodiments, the NK ICVs are loaded with one or more anticancer or chemotherapeutic agents. In yet a further embodiment of any of the foregoing embodiments, wherein the one or more anticancer or chemotherapeutic agents are selected from the group of doxorubicin, daunorubicin, all-trans retinoic acid, mitoxantrone, podocalyxin, paclitaxel, and any combination thereof.

In a particular embodiment, the disclosure further provides for chimeric antigen receptor natural killer cell induced cellular vesicles (CAR-NK ICVs) produced by a method disclosed herein. In a further embodiment, the CAR-NK ICVs are loaded with one or more small molecule therapeutic compounds or agents. In yet a further embodiment of any of the foregoing embodiments, the CAR-NK ICVs are loaded with one or more anticancer or chemotherapeutic agents. In yet a further embodiment of any of the foregoing embodiments, the one or more anticancer or chemotherapeutic agents are selected from the group of doxorubicin, daunorubicin, all-trans retinoic acid, mitoxantrone, podocalyxin, paclitaxel, and any combination thereof.

In a certain embodiment, the disclosure provides for a pharmaceutical composition comprising a pharmaceutically acceptable carrier and NK ICVs or CAR-NK ICVs disclosed herein.

In a particular embodiment, the disclosure also provides a method for treating a subject with cancer, comprising administering a therapeutically effective amount of the pharmaceutical composition disclosed herein to a subject in need thereof. In a further embodiment, the NK ICVs or CAR-NK ICVs are produced from autologous natural killer cells of the subject to be treated.

### DESCRIPTION OF DRAWINGS

FIG. 1A-B provides microscope images of induced cellular vesicle production of NK92 cells over time, after exposure to (A) paraformaldehyde and dithiothreitol, or (B) N-ethylmaleimide. Arrows indicate induced cellular vesicles.

FIG. 2 demonstrates dose-dependent toxicity of NK92 ICVs with K562 Leukemia cells as assessed by an MTT assay.

FIG. 3A-B demonstrates dose-dependent toxicity of NK92 nICVs and mICVs with HeLa cervical cancer cells as assessed by an MTT assay. (A) NK92 ICVs induced with PFA; (B) NK92 ICVs induced with NEM.

FIG. 4 demonstrates dose-dependent toxicity of NEM induced NK92 ICVs with MCF7 cancer cells as assessed by an MTT assay.

FIG. 5 demonstrates that chemotherapeutics can be loaded into NK ICVs to enhance toxicity. Nano-scale ICVs were produced and isolated from NK92 cells or K562 cancer cells. ICVs were then loaded with doxorubicin (DOX), a cancer therapeutic.

FIG. 6 presents a diagram showing a sampling of major NK receptors that can be found on the surface of NK ICVs. NKp46, natural killer cell p46-related protein; NKp44, natural killer cell p44-related protein; NKp30, natural killer cell p30-related protein; CD, Cluster of differentiation; NKG2, also known as CD159; KIR, killer-cell immunoglobulin-like receptor; TIGIT, T cell immunoreceptor with Ig and ITIM domains; LAG3, lymphocyte activation gene 3 protein; TIM3, T cell immunoglobulin mucin receptor 3; PD1, programmed cell death protein 1; KLRG1, killer cell lectin-like receptor subfamily G member 1; IL-2R, interleukin-2 receptor; TGFβR, transforming growth factor beta receptors. The figure further shows that the NK ICV can be loaded with anticancer agent(s).

FIG. 7 presents that various generations of chimeric antigen receptor (CAR) design that can be applied to NK ICVs. The traditional CAR vector structure consists of three parts: an extracellular antigen recognition region, a transmembrane region, and an intracellular signal domain. The extracellular domain of CAR includes an scFv region (H [heavy] and L [light] chain) that is spliced by a linker. A hinge ensures flexibility and connects to the transmembrane domain. The intracellular domain includes a CD3ζ signaling domain and costimulatory domains, such as CD28, CD137, and 2B4.

### DETAILED DESCRIPTION

As used herein and in the appended claims, the singular forms “a,” “an,” and “the” include plural referents unless the context clearly dictates otherwise. Thus, for example, reference to “an induced cellular vesicle” includes a plurality of such induced cellular vesicles and reference to “the sulfhydryl blocking agent” includes reference to one or more sulfhydryl blocking agents and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments only and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

The terms “blebbing”, “plasm membrane blebbing” or “cell membrane blebbing” as used herein, all refer to methods disclosed herein that induce plasma membrane blebbing in cells resulting in the production of induced cellular vesicles. Typically, blebbing of the plasma membrane is a morphological feature of cells undergoing late stage apoptosis. A bleb is an irregular bulge in the plasma membrane of a cell caused by localized decoupling of the cytoskeleton from the plasma membrane. The bulge eventually separates from the parent plasma membrane taking part of the cytoplasm with it to form a vesicle. Blebbing is also involved in some normal cell processes, including cell locomotion and cell division. Cell blebbing can be manipulated by mechanical or chemical treatment. It can be induced following microtubule disassembly, by inhibition of actin polymerization, increasing membrane rigidity or inactivating myosin motors, and by modulating intracellular pressure. Induced cellular vesicles can also be induced in response to various extracellular chemical stimuli, such as exposure to agents that bind up sulfhydryl groups (i.e., sulfhydryl blocking agents).

The term “blebbing agent”, as used herein refers to chemical agents, such as sulfhydryl blocking agents, that when administered to cells induce the cells to undergo plasma membrane blebbing.

The term “sulfhydryl blocking agent” as used herein, refers to compound or reagent that interacts with cellular sulfhydryl groups so that the sulfhydryl group is blocked or bound up by the sulfhydryl blocking agent, typically via alkylation or disulfide exchange reactions. Chemical agents that can be used in the methods or compositions disclosed herein that block or bind up sulfhydryl groups includes, but are not limited to, mercury chloride, p-chloromercuribenzene sulfonic acid, auric chloride, p-chloromercuribenzoate, chlormerodrin, meralluride sodium, iodoacetamide, paraformaldehyde, dithiothreitol and N-ethylmaleimide.

The term “a sulfhydryl blocking agent that induces cellular vesicle production” or the like as used herein, refers to a small molecule compound that when administered induces plasma membrane blebbing in cells, usually by causing injuries to cells by binding up or blocking sulfhydryl groups of biomolecules, such as proteins.

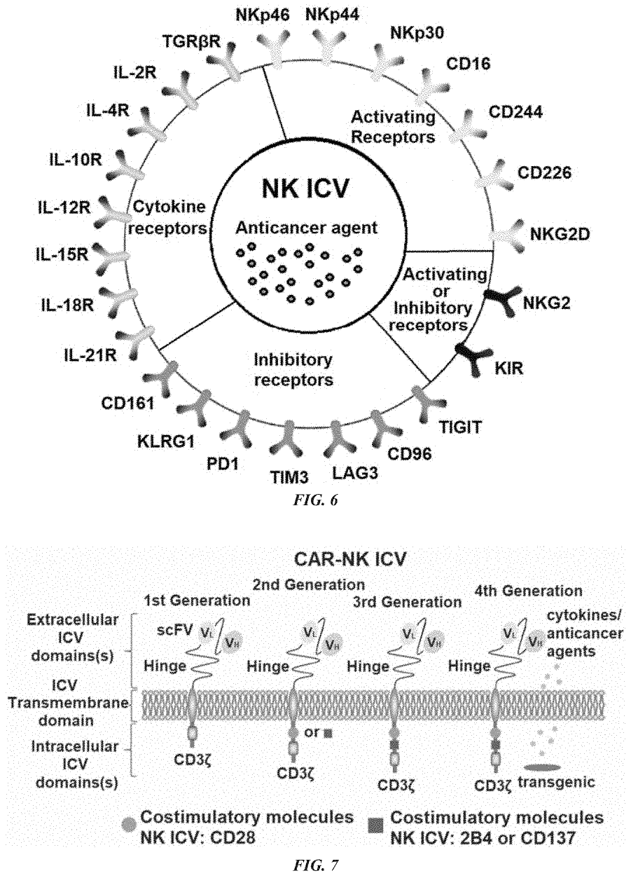

The term “natural killer cell” or “NK cell” refers to a type of lymphocyte (a white blood cell), a component of the innate immune system. NK cells play a major role in the host-rejection of both tumors and virally infected cells. NK cells have many types of receptors on the cell surface that have different functions (see FIG. 6). NK cells are cytotoxic; small granules in their cytoplasm contain special proteins such as perforin and proteases known as granzymes. Upon release in close proximity to a cell slated for killing, perforin forms pores in the cell membrane of the target cell through which the granzymes and associated molecules can enter, inducing apoptosis. For purposes of this disclosure “natural killer cell” or “NK cell” are preferably of human origin, and can include cells isolated from a subject, preferably a human subject, or NK-based cell lines, such as NK-92 and NK-92MI cells lines. NK cells can be isolated from peripheral blood mononuclear cells or washed leukapheresis samples using commercially available kits (e.g., see EasySep™ Human NK Cell Isolation Kit by STEMCELL; NK Cell Isolation Kit by Miltenyi Biotec) or can be isolated using various protocols described in the art (e.g., Ferlazzo G., Methods Mol Biol. 415:197-213 (2008); and Pak-Wittel et al., Curr Protoc Immunol. 105(1): 3.22.1-3.22.9 (April 2014)).

The term “natural killer induced cellular vesicle” or “NK ICV” as used herein, refers to an induced cellular vesicle that is produced by a natural killer cell as the direct result of the use of a blebbing agent as described herein. For purposes of this disclosure, “natural killer induced cellular vesicle” or “NK ICV” includes genetically or phenotypically modified NK ICVs, like CAR-NK ICVs, unless indicated otherwise. The methods and compositions described herein can be applied to NK ICVs of all sizes. In a particular embodiment, the method and compositions described herein comprise NK ICVs that have an average diameter of 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, 1100 nm, 1200 nm, 1300 nm, 1400 nm, 1500 nm, 1600 nm, 1700 nm, 1800 nm, 1900 nm, 2000 nm, 2500 nm, 3000 nm, 3500 nm, 4000 nm, 5000 nm, 10 μm, 15 μm, 20 μm, 30 μm, 40 μm, 50 μm, 60 μm, 70 μm, 80 μm, 90 μm, 100 μm, or any range that includes or is between any two of the foregoing values, including fractional increments thereof. Moreover, the NK ICVs disclosed herein may be used to encapsulate a biological molecule, such as nucleic acids, proteins, peptides, lipids, oligosaccharides, etc.; therapeutic agents, such as drug products like chemotherapeutic agents; prodrugs; gene silencing agents; chemotherapeutics; diagnostic agents; and components of a gene editing system, such as the CRISPR-Cas system, a CRISPRi system, or CRISPR-Cpf1 system, etc. In a particular embodiment, a NK EB disclosed herein encapsulates a chemotherapeutic or anticancer agent.

The terms “natural killer nanometer sized induced cellular vesicle”, or “NK nICV” as used herein, refer to induced cellular vesicles produced by natural killer cells using a blebbing agent as described herein having a dimeter in the nanometer size range. In a particular embodiment, the NK nICV has a diameter of 5 nm, 10 nm, 20 nm, 30 nm, 40 nm, 50 nm, 60 nm, 70 nm, 80 nm, 90 nm, 100 nm, 150 nm, 200 nm, 250 nm, 300 nm, 400 nm, 500 nm, 600 nm, 700 nm, 800 nm, 900 nm, up to 1000 nm, or is a range that includes or is between any two of the foregoing values, including fractional increments thereof.

The terms “natural killer micrometer sized induced cellular vesicle”, or “NK mICV” as used herein, all refer to induced cellular vesicles produced by natural killer cells using a blebbing agent as described herein having a dimeter in the micrometer size range. In a particular embodiment, the NK mICV has a diameter of 1 μm, 2 μm, 3 μm, 4 μm, 5 μm, 10 μm, 15 μm, 20 μm, 30 μm, 40 μm, 50 μm, 60 μm, 70 μm, 80 μm, 90 μm, 100 μm, or a range that includes or is between any two of the foregoing values, including fractional increments thereof.

For purposes of the disclosure the term “cancer” will be used to encompass cell proliferative disorders, neoplasms, precancerous cell disorders and cancers, unless specifically delineated otherwise. Thus, a “cancer” refers to any cell that undergoes aberrant cell proliferation that can lead to metastasis or tumor growth. Exemplary cancers include but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic my elogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

The term “effective amount” as used herein, refers to an amount that is sufficient to produce at least a reproducibly detectable amount of the desired result or effect. An effective amount will vary with the specific conditions and circumstances. Such an amount can be determined by the skilled practitioner for a given situation.

The terms “patient”, “subject” and “individual” are used interchangeably herein, and refer to an animal, particularly a human, to whom treatment including prophylaxis treatment is provided. This includes human and non-human animals. The term “non-human animals” and “non-human mammals” are used interchangeably herein includes all vertebrates, e.g., mammals, such as non-human primates, (particularly higher primates), sheep, dog, rodent (e.g., mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. “Mammal” refers to any animal classified as a mammal, including humans, non-human primates, domestic and farm animals, and zoo, sports, or pet animals, such as dogs, cats, cattle, horses, sheep, pigs, goats, rabbits, etc. Patient or subject includes any subset of the foregoing, e.g., all of the above, but excluding one or more groups or species such as humans, primates or rodents. A subject can be male or female. A subject can be a fully developed subject (e.g., an adult) or a subject undergoing the developmental process (e.g., a child, infant or fetus).

The term “purified” when used in reference to an NK ICV disclosed herein, refers to the fact that the ICV is removed from the majority of other cellular components from which it was generated or in which it is typically present in nature. The ICVs disclosed herein are typically prepared to the state where they are purified or semi-purified.

The term “therapeutically effective amount” as used herein, refers to an amount that is sufficient to affect a therapeutically significant reduction in one or more symptoms of the condition when administered to a typical subject who has the condition. A therapeutically significant reduction in a symptom is, e.g., about 10%, about 20%, about 30%, about 40%, about 50%, about 60%, about 70%, about 80%, about 90%, about 100%, or more as compared to a control or non-treated subject.

The term “treat” or “treatment” as used herein, refers to a therapeutic treatment wherein the object is to eliminate or lessen symptoms. Beneficial or desired clinical results include, but are not limited to, elimination of symptoms, alleviation of symptoms, diminishment of extent of condition, stabilized (i.e., not worsening) state of condition, delay or slowing of progression of the condition.

The development and progression of cancer is promoted by unbalanced homeostasis in the growth of cells, which is largely attributed to the suppression of apoptotic pathways and the increased expression of survival pathways. As cancer cells progress into tumors, a patient's own immune system can recognize and destroy these abnormal cells using natural killer (NK) cells. Upon recognition of abnormal cells, NK cells form an immunological synapse and release cytotoxic granules to the target cell, inducing cell death. NK cell cytotoxicity is also regulated by recognition of self-associated molecules (such as major histocompatibility complex or MHC class I), which inhibit cytotoxic granule release to non-target cells. In this way, NK cells can act as targeted and effective therapeutics for cancer.

However, many cancers often grow and metastasize at a rate and scale that cannot be managed by the immune system alone. To boost NK availability, recent clinical trials have investigated and proven that a leukemia patient's own NK cells can be harvested, expanded ex vivo and used to treat the patient. While NK cell therapy is promising, using whole cells implies inherent risks, poorly defined good manufacturing protocols, and poor storage capability.

It was postulated herein that NK cell-derived extracellular vesicles could be a promising solution to maintain the inherent bio-active nature of NK cells with fewer risks and a more stable design for transport and storage. Extracellular Vesicles (EVs) have shown great promise as drug delivery carriers due to their unique advantages resulting from intrinsic biocompatibility. Exosomes, 30-100 nm EVs released from multi-vesicular cytoplasmic bodies, are the most widely studied EVs for therapeutic delivery. Cells naturally utilize EVs for transporting mRNA and microRNA between cells, and therapeutic-loaded EVs have been used to achieve tissue-specific delivery of exogenous RNA and drugs. EVs are promising cell-derived vesicles that are compatible with patients' bodies, loadable with a broad range of therapeutics, and capable of generating synergistic therapies through the cell's specific and multidimensional functions. EVs can generally be used to encapsulate cargo from their parent cells and have a wide range of biological functions.

The use of EV based therapies, however, has been plagued with low EV yields and a lack of homogeneity in regards to EV size and compositions. Extracellular Vesicle production from NK cells also suffer from low production yields and a lack of homogeneity. Faced with the foregoing limitations, extracellular vesicles from natural killer cells have found limited use as a cancer treatment option.

Provided herein are methods that address the foregoing limitations with EV production by employing a unique and efficient chemically-induced production technique that initiates rapid blebbing of the NK cell membrane. Contrary to other NK extracellular vesicle production techniques, the methods of the disclosure rapidly generate high yields of NK induced cellular vesicles (ICVs) that are identical in presentation to their parent cells. Thus, the ICVs of the disclosure are structurally distinguishable from EVs normally produced by NK cells. The ICV production technique disclosed herein presents a scalable option for producing cell-free NK ICV cancer therapeutics that has industrial and medicinal applicability. Moreover, by using the blebbing agents described herein, NK cells can be induced to produce nano- and micro-scale ICVs that can be used as cancer therapeutics. By maintaining the bioactive properties of NK cells, ICVs can target cancer cells and inherently avoid release of cytotoxic payloads to normal cells, reducing toxic side effects. Furthermore, NK ICVs can be loaded with other chemotherapeutic agents to increase toxicity while maintaining specificity.

Natural killer (NK) cell activity was first described in mice in 1964 as activity in which lethally irradiated mice without prior sensitization could resist BM allografts. In humans, NK cells express the adhesion marker CD56 and lack the TCR CD3. They are derived from CD34⁺ progenitor cells in the BM and migrate upon differentiation to lymphoid tissue and peripheral blood. IL-15 is essential for NK cell development and homeostasis because IL-15-knockout mice lack NK cells. Furthermore, IL-15 activity is enhanced when trans-presented by IL-15 receptor alpha on cells such as dendritic cells. Blood NK cells can be divided on the basis of surface density of CD56 into CD56^{bright} and CD56^{dim} NK cells. Resting CD56^{bright} regulatory NK cells are more proliferative, produce high levels of cytokines, and are poor mediators of NK cell natural cytotoxicity. In contrast, CD56^{dim} NK cells are potently cytotoxic and mediate antibody-dependent cellular cytotoxicity (ADCC) through CD16 (FcγRIII) without cytokine activation. NK cells produce a wide variety of cytokines and chemokines such as IFNγ, G-CSF, TNFα, TGF-β, macrophage inflammatory protein 1-beta (MIP-1β), and RANTES. NK cells can express inhibitory receptors for both self- and non-self MHC class I molecules. NK cell cytotoxicity is triggered by the loss of MHC class I on the tumor cells. Different families of receptors were also identified on NK cells that recognize MHC class I to mediate tolerance in the host. Because of their ability to lyse tumors with aberrant MHC class I expression and to produce cytokines and chemokines upon activation, NK cells have great therapeutic potential to treat cancer and enhance the benefits of hematopoietic cell transplantation. Promising data suggest that NK cells are effective at preventing relapse or treating acute myeloid leukemia (AML) and ongoing trials are under way in many other disease settings.

Among the several NK cell lines currently established, NK-92 cells lack almost all inhibitory killing receptors and express a series of activating receptors. Additionally, NK-92 cells are abundant in perforin and granzyme, suggesting a broad spectrum of cytotoxic effects. NK-92 cells were found to be cytotoxic to leukemia cells both in vitro and in vivo. NK-92 cells were further found to effectively kill clonogenic and bulk multiple myeloma cells and could significantly reduce tumor burden in vivo. NK-92 cells are approved for analysis in clinical trials to determine their utility in the treatment of some types of malignant tumors. The studies presented herein indicate that ICVs can be produced from NK-92 cells, and other NK cell lines, by using the blebbing methods of the disclosure. As in vitro studies with NK-92 cells are established as being correlative to results obtained in vivo, the studies indicate that the cancer killing properties of the NK ICVs disclosed herein support their use as therapeutics for treating cancer in vivo.

Further, investigators have shown that chimeric antigen receptor NK 92 cells (CAR-NK-92 cells) can be used in manner similar to chimeric antigen receptor T-cells (CAR-T cells), but do not suffer from the same drawbacks. CAR-T cells have been approved by the Federal Drug Commission for use in relapsed and refractory B cell malignancies by targeting CD19. However, there are many limitations to the use of CAR-T cells, including off-target effects and cytokine storms. Therapy with CAR-T cells has not yet been successful in patients with solid tumors, and the production of autologous cells also has many limitations in the clinical setting. The production of CAR-T cells requires a certain time period, which makes it challenging to prepare a sufficient number of CAR-T cells within a short time for patients whose disease progresses faster. It is also difficult to collect a sufficient number of healthy T cells from the patient. “Off-the-shelf” allogeneic T cells can overcome these difficulties, but may cause severe graft-versus-host disease (GVHD).

CAR-NK-92 cells have been found to be highly cytotoxic and can be harvested as phenotypically homogeneous cells, with production of large numbers of cells within a short period. Additionally, compared with CAR-T cells, CAR-NK-92 cells cannot proliferate after irradiation; thus, the survival time in vivo is shorter, avoiding some off-target effects. Even if the targeted antigen on the tumor is rapidly lost, the CAR-NK-92 cells can still be activated by their activating receptors, conferring CAR-NK-92 cells with significant advantages. Similar to CAR-T cells, CAR-NK cells provide a new activation pathway to enhance the antitumor effects of the cells and to improve tumor cell targeting. CAR-NK ICVs produced from CAR-NKs have the basic framework of CAR-NKs (see FIG. 7), including an extracellular antigen recognition region, a transmembrane region, and an intracellular signal domain. The CAR constructs used in making CAR-NKs are composed of an extracellular antigen-recognition ScFv connected via a flexible linker to a transmembrane domain followed by an intracellular signaling/activation CD3ζ domain that provides a signal to activate NK cells. CARs have evolved through the addition of costimulatory molecules to the intracellular CD3ζ to enhance cytotoxicity and durability. In the case of 1^{st} generation CARs, a cluster of differentiation CD3ζ chain-derived signaling domain are responsible for effector cell activities while 2^{nd} and 3^{rd} generation CARs have one or two costimulatory domains, respectively, to boost CD3ζ function (see FIG. 7). In addition to engineered cancer-homing receptors, CAR-expressing NK cells retain their native NK cell receptor-dependent mechanisms, making them potent effectors for immunotherapy as described in Hu et al., Acta Pharmacol Sin. 39(2):167-176 (2018).

CD3ζ is a classical intracellular signal segment of the CAR structure and plays an important role in NK cells. CAR-NK generally uses CD3ζ as the first signal motif (first-generation CAR) and then a costimulatory molecular motif (second-generation CAR), such as CD28 or CD137 (4-1BB), to form an intracellular signal region. NKG2D is an important activating receptor expressed on most CD8⁺ T cells and NK cells and is a relatively unique activating receptor in NK cells.

The NKG2D receptor binds to DAP10 or DAP12 transfer proteins to provide different activation signals. Both signals can activate the cytotoxicity of NK cells, but only the activation signal transmitted by DAP12 can promote the production of cytokines by NK cells. In one study, researchers linked DAP10 and CD3ζ to the NK cell activation receptor NKG2D. In an osteosarcoma mouse model, the cytotoxic potential of NK cells against a wide spectrum of tumor subtypes could be markedly enhanced by expression of CAR-NKG2D-DAP10-CD3ζ receptor. CD244, also known as the NK cell receptor 2B4, is a signal transduction lymphocyte-activating molecule-related receptor expressed in all NK cells. This protein is an important regulator of NK cell activation and was shown to have robust costimulatory roles in a study in which NK cells were used as effector cells to target CD19 or GD2.

NK-92 cells are an ideal CAR carrier with natural antitumor properties and are easy to cultivate and modify in vitro. The first generation of CAR has been widely applied in CAR-NK-92 cells. CAR-NK-92 cells do not cause GVHD and have greater cytotoxicity than ADCC. Indeed, CAR-NK-92 cells have many advantages, as follows: (1) CAR-NK-92 cells can target tumor cells and directly activate NK-92 cells to kill target cells; (2) even if the targeted antigen on the tumor is rapidly lost, the CAR-NK-92 cells can still be activated by their activating receptors; and (3) the inhibitory receptors are expressed at low levels on the surface and deletion of inhibitory receptors makes NK-92 cells more resistant to solid tumors than other immune cells. For safety reasons, NK-92 cells are lethally irradiated before clinical application. After irradiation, CAR-NK-92 cells survive in vivo for a short period time. The antitumor ability of NK cells confers them with broad potential applications in cell therapy. CAR-NK-92 cells have also been shown to be promising as effector cells. Current therapies using CAR-NK cells include those presented in Table 1.

TABLE 1
List of active clinical trials involving CAR-NK cells worldwide.

| Clinical | | | NK Cell | | |
|---|---|---|---|---|---|
| Trial # | Target Antigen | Disease | Source | Phase | Sponsor |
| NCT02742727 | CD7 | Lymphoma | NK-92 cell | I/II | PersonGen BioTherapeutics |
| | | Leukemia | line | | (Suzhou) Co., Ltd. |
| NCT02839954 | MUC1 | Solid tumors | NK-92 cell | I/II | PersonGen BioTherapeutics |
| | | | line | | (Suzhou) Co., Ltd. |
| NCT02892695 | CD19 | Lymphoma | NK-92 cell | I/II | PersonGen BioTherapeutics |
| | | Leukemia | line | | (Suzhou) Co., Ltd. |
| NCT02944162 | CD33 | Acute myeloid | NK-92 cell | I/II | PersonGen BioTherapeutics |
| | | leukemia | line | | (Suzhou) Co. |
| NCT03056339 | CD19 | Lymphoma | UCB | I/II | MD Anderson Cancer Center |
| | | Leukemia | | | |
| NCT03383978 | HER2 | Glioblastoma | NK-92 cell | I | Johann Wolfgang Goethe |
| | | | line | | University Hospital |
| NCT03415100 | NKHZD ligands | Solid tumors | Autologous or | I | The Third Alfiliated Hospital |
| | | | allogeneic NK cells | | of Guangzhou Medical |
| | | | | | University |
| NCT03579927 | CD19 | Lymphoma | UCB | I/II | Md Anderson Cancer Center |
| | | Leukemia | | | |
| NCT03656705 | — | Non-small cell | Modified NK-92 | I | XinXiang Medical University |
| | | lung cancer | cell line | | |
| NCT03841110 | — | Solid | iPSC-derived CAR- | I | Fate Therapeutics |
| | | tumors | NK cells (FT500) | | |

NK ICVs may be produced from NK cells and genetically modified NK cells (e.g., CAR-NKs) by contacting the cells with a chemical agent that induces blebbing as further described herein. The NK ICVs can be produced from an immortalized NK cell line, such as NK-92, NK-92MI, NKL, KYHG-1, and NKG. Alternatively, the NK ICVs can be produced from NK cells that have been differentiated from stem cells or progenitor cells. Human embryonic stem cells (hESCs) and induced pluripotent stem cells (iPSCs) can offer another renewable and potentially better source of NK cells. These undifferentiated cells can be culture expanded and subsequently differentiated into NK cells as described by Dahlberg et al., Front Immunol. 6:605 (2015) and Zeng et al., Stem Cell Reports 9(6):1796-1812 (2017). Both iPSC-derived NK cells and those from NK cell lines have the advantage that they can be extensively tested and characterized to maintain specific standards and offers the unique opportunity to manufacture CAR-NK cells under Good Manufacturing Practices (GMP) guidelines. Alternatively, NK cells can be isolated as primary cells from a multicellular organism, in particular, a human. The primary cells may be isolated and used as is, or may be grown or propagated in the laboratory for a short period of time (e.g., 10 or fewer passages, 50 or fewer passages, 100 or fewer passages). Further, the primary cell may be NK cells obtained from a subject to be treated, i.e., personalized treatment. In other words, the subject is treated with NK ICVs that are produced from the subject's own natural killer cells. Additionally, NK cells and genetically modified NKs are commercially available. For example, CAR-NKs are available from Promab.

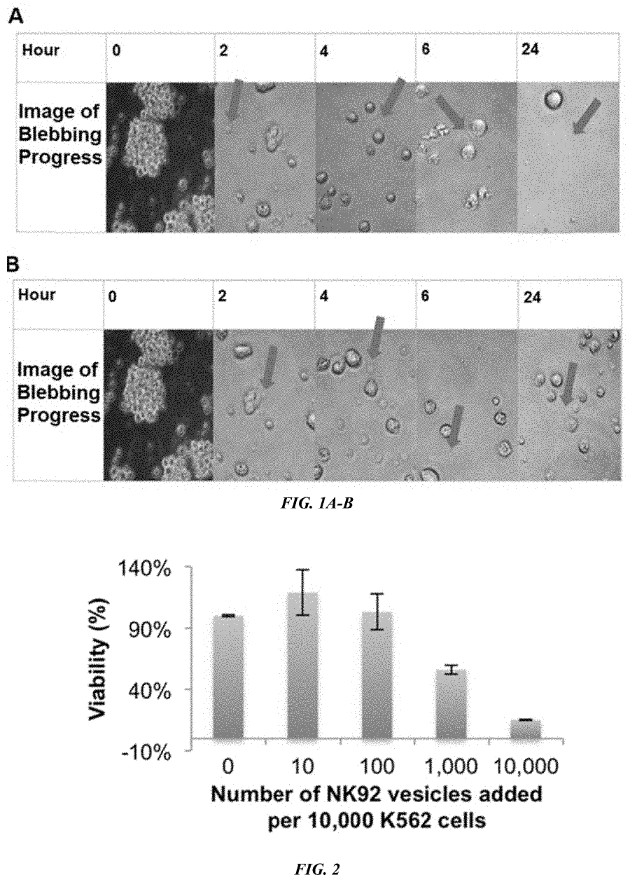

In particular, the disclosure provides for techniques and methods that provide for high yields of NK ICVs in as little as a few hours, producing both micro and nano-scale sized NK ICVs. For example, use of the blebbing agents described herein can induce the production of NK ICVs in as little as 2-5 h (e.g., see FIG. 1). Further, in the studies provided herein, the NK ICVs exert a dose dependent cytotoxic effect when they were administered to a variety of different types of cancer cells (e.g., see FIG. 2-5). The cytotoxic effect of the NK ICVs can be further modulated based upon the size of the NK ICVs used, and the choice of blebbing agent (e.g., see FIG. 2-5).

In a further embodiment, the chemical agent that induces blebbing is a sulfhydryl blocking agent. Examples of sulfhydryl blocking agents include, but are not limited to, mercury chloride, p-chloromercuribenzene sulfonic acid, auric chloride, p-chloromercuribenzoate, chlormerodrin, meralluride sodium, iodoacetmide, paraformaldehyde, dithiothreitol, and N-ethylmaleimide. In a particular embodiment, NK ICVs are produced from blebbing induced in natural killer cells by contacting the natural killer cells with (1) paraformaldehyde, (2) paraformaldehyde and dithiothreitol, or (3) N-ethylmaleimide. In a further embodiment, NK ICVs are produced from blebbing induced in natural killer cells by contacting the natural killer cells with a solution comprising paraformaldehyde at of 20 mM, 25 mM, 30 mM, 35 mM, 40 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, or a range that includes any two of the foregoing concentrations, including from 20 mM and 250 mM, and from 25 mM to 50 mM.

In a yet a further embodiment, the solution comprising paraformaldehyde (PFA) further comprises dithiothreitol (DTT) at concentration of 0.2 mM, 0.4 mM, 0.5 mM, 0.6 mM, 0.8 mM, 1 mM, 1.1 mM, 1.2 mM, 1.3 mM, 1.4 mM, 1.45 mM, 1.5 mM, 1.55 mM, 1.6 mM, 1.65 mM, 1.7 mM, 1.75 mM, 1.8 mM, 1.85 mM, 1.9 mM, 1.95 mM, 2.0 mM, 2.1 mM, 2.2 mM, 2.3 mM, 2.4 mM, 2.45 mM, 2.5 mM, 2.55 mM, 2.6 mM, 2.65 mM, 2.7 mM, 2.75 mM, 2.8 mM, 2.85 mM, 2.9 mM, 2.95 mM, 3.0 mM, 3.1 mM, 3.2 mM, 3.3 mM, 3.4 mM, 3.45 mM, 3.5 mM, 3.55 mM, 3.6 mM, 3.65 mM, 3.7 mM, 3.75 mM, 3.8 mM, 3.85 mM, 3.9 mM, 3.95 mM, 4.0 mM, 4.5 mM, 5.0 mM, 5.5 mM, 6.0 mM, 6.5 mM, 7.0 mM, 7.5 mM, 8.0 mM, 8.5 mM, 9.0 mM, 9.5 mM, 10 mM, or any range that includes or is between any two of the foregoing concentrations, including from 1.0 mM to 3 mM, and 1.5 mM to 2.5 mM. In an alternate embodiment, NK ICVs are produced from blebbing induced in natural killer cells by contacting the natural killer cells with a solution comprising N-ethylmaleimide (NEM) at concentration of 0.2 mM, 0.4 mM, 0.5 mM, 0.6 mM, 0.8 mM, 1.0 mM, 1.5 mM, 2.0 mM, 2.5 mM, 3.0 mM, 3.5 mM, 4.0 mM, 4.5 mM, 5.0 mM, 5.5 mM, 6.0 mM, 6.5 mM, 7.0 mM, 7.5 mM, 8.0 mM, 8.5 mM, 9.0 mM, 9.5 mM, 10.0 mM, 10.5 mM, 11.0 mM, 11.5 mM, 12 mM, 12.5 mM, 13.0 mM, 13.5 mM, 14.0 mM, 14.5 mM, 15.0 mM, 15.5 mM, 16.0 mM, 16.5 mM, 17.0 mM, 17.5 mM, 18.0 mM, 18.5 mM, 19.0 mM, 19.5 mM, 20.0 mM, or any range that includes or is between any two of the foregoing concentrations, including from 2.0 mM to 20.0 mM, and 2.0 mM to 5.0 mM. In a further embodiment, the solution comprising PFA; PFA and DTT; or NEN comprises a buffered balanced salt solution. Examples of buffered saline solutions include but are not limited to, phosphate-buffered saline (PBS), Dulbecco's Phosphate-buffered saline (DPBS), Earles's Balanced Salt solution (ICVSS), Hank's Balanced Salt Solution (HBSS), TRIS-buffered saline (TBS), and Ringer's balanced salt solution (RBSS). In a further embodiment, the solution comprising PFA; PFA and DTT; or NEN comprises a buffered balanced salt solution at a concentration/dilution of 0.5×, 0.6×, 0.7×, 0.8×, 0.9×, 1×, 2×, 3×, 4×, 5×, 6×, 7×, 8×, 9×, and 10×, or any range that includes or is between any two of the foregoing concentrations/dilutions, including fractional values thereof.

In a certain embodiment, the disclosure also provides that the NK ICVs may be collected by any suitable means to separate NK ICVs from NK cells or NK cell debris. In some embodiments, to isolate NK ICVs, cells and cell debris can be removed by centrifugation at 1000 to 1500 rpm for 1, 1.5, 2, 2.5, 3, 3.5, 4, 4.5, 5, 5.5, 6, 6.5, 7, 7.5, 8, 8.5, 9, 9.5, or 10 minutes followed by removal of NKs and NK cell debris. NK mICVs and nICVs can then be recovered by centrifugation at 10,000×g to 18,000×g for 5, 6, 7, 8, 9, 10, 11, 12, 13, 14, or 15 minutes. NK ICVs be further concentrated using concentrators. The size of the NK ICVs disclosed herein could be controlled by using the isolation methods presented herein.

In a particular embodiment, NK cells can be phenotypically or genetically modified, such as for producing CAR-NK cells. The NK ICVs can then be produced from these genetically modified NK cells. Virus transduction is the most common method used for genetic modification of NK cells. These viral vectors include retroviral vectors, lentiviral vectors, associated adenoviral vectors and adenoviral vectors, among which retroviral vectors and lentiviral vectors are most widely used. Viral vectors are capable of ensuring stable expression of the transgene. For example, NK cells can be transduced to express CARs for cancer retargeting in much the same way as in T cells. Viral vectors for modifying T-cells to express CAR are commercially available (e.g., Creative-Biolabs). Nonviral vectors that use genome editing like Zinc-finger nucleases, TALEN, and CRISPR-Cas9 systems can be combined with cell transfection techniques to precisely insert genes into the genome and achieve stable expression of CARs. A non-viral Sleeping Beauty (SB) transposon system may also be used to generate stable transgene expression but without the risks associated with viral vectors.

The disclosure further provides that the NK ICVs disclosed herein may be used (1) in combination with other agents or molecules, and/or (2) loaded with other agents or molecules, such as biological molecules, therapeutic agents, prodrugs, gene silencing agents, chemotherapeutics, adjuvants, diagnostic agents, and/or components of gene editing systems. In a particular embodiment, the NK ICVs are used in combination with or loaded with a cargo comprising one or more anticancer agents. Examples of anticancer agents, include, but are not limited to, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and tiimethylolomelamine; acetogenins (e.g., bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; vinca alkaloids; epipodophyllotoxins; antibiotics such as the enediyne antibiotics (e.g., calicheamicin, especially calicheamicin gammall and calicheamicin omegall; L-asparaginase; anthracenedione substituted urea; methyl hydrazine derivatives; dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitiaerine; pentostatin; phenamet; pirarubicin; losoxantione; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2 2″-trichlorotiiethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® (docetaxel) (Rhone-Poulenc Rorer, Antony, France); chloranbucil; GEMZAR® (gemcitabine); 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DFMO); retinoids such as retinoic acid; capecitabine; leucovorin (LV); irenotecan; adrenocortical suppressant; adrenocorticosteroids; progestins; estrogens; androgens; gonadotropin-releasing hormone analogs; and pharmaceutically acceptable salts, acids or derivatives of any of the above. Also included anticancer agents are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASL® exemestane, formestanie, fadrozole, RIVISOR® vorozole, FEMARA® letrozole, and ARTMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF-A expression inhibitor (e.g., ANGIOZYME® ribozyme) and a HER2 expression inhibitor; vaccines such as gene therapy vaccines, for example, ALLOVECTINO vaccine, LEUVECTIN® vaccine, and VAXID® vaccine; PROLEUKIN® rJL-2; LURTOTECAN® topoisomerase 1 inhibitor; ABARELLX® rmRH; antibodies such as trastuzumab and pharmaceutically acceptable salts, acids or derivatives of any of the above.

NK ICVs produced in accordance with embodiments of the disclosure may also be loaded with the cargo via direct membrane penetration, chemical labeling and conjugation, electrostatic coating, adsorption, absorption, electroporation, or any combination thereof. Further, NK ICVs produced in accordance with certain embodiments of the disclosure may undergo multiple loading steps, such that some cargo may be introduced to NKs prior to blebbing, while additional cargo may be loaded during or after blebbing. Additionally, NK ICVs may be loaded with the cargo during blebbing, and further loaded with another cargo after blebbing. In a further embodiment, the NK ICVs may be loaded with a cargo as defined above by incubating NKs cells or NK ICVs with a cargo having the concentration of 25 pg/mL, 50 pg/mL, 100 pg/mL, 200 pg/mL, 300 pg/mL, 400 pg/mL, 500 pg/mL, 600 pg/mL, 700 pg/mL, 800 pg/mL, 900 pg/ml, 1 ng/mL, 10 ng/mL, 100 ng/mL, 1 pg/mL, 10 ug/mL or any range that includes or is between any two of the foregoing concentrations. Additionally, the incubation may occur for 1 hour, 2 hours, 3 hours, 4 hours, 5 hours, 6 hours, 12 hours, 24 hours, 48 hours, or any range that includes or is between any two of the foregoing time points. Alternatively, the loading conditions may occur at a ratio of NK ICVs to a compound of 1:20 to 20:1, 1:15 to 15:1, 12:1 to 1:12, 11:1 to 1:11, 10:1 to 1:10, 9:1 to 1:9, 8:1 to 1:8, 7:1 to 1:7, 6:1 to 1:6, 5:1 to 1:5, 4:1 to 1:4, 3:1 to 1:3, 2:1 to 1:2, 1.5:1 to 1:1.5, or 1:1. Additionally, the polydispersity of cargo-loaded NK ICVs may have a similar polydispersity index (PDI) as unloaded NK ICVs. As such, cargo-loaded NK ICVs may have a PDI of about 0.1, 0.2, 0.3, 0.4, 0.5, 0.6, 0.7, 0.8, 0.9, 1.0, or any range that includes or is between any two of the foregoing values.

The disclosure further provides for pharmaceutical compositions and formulations comprising NK ICVs described herein for specified modes of administration. In one embodiment, a pharmaceutical composition comprises NK ICVs and a pharmaceutically acceptable carrier. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the composition and is compatible with administration to a subject, for example a human. Such compositions can be specifically formulated for administration via one or more of a number of routes, such as the routes of administration described herein. Supplementary active ingredients also can be incorporated into the compositions. When an agent, formulation or pharmaceutical composition described herein, is administered to a subject, preferably, a therapeutically effective amount is administered. As used herein, the term “therapeutically effective amount” refers to an amount that result in an improvement or remediation of the condition.

The disclosure further provides for the use of a pharmaceutical composition comprising NK ICVs for the treatment of a subject having or suspected of having cancer. In a further embodiment, the disclose also provides methods for treating a subject having cancer comprising: administering a therapeutically effective amount of NK ICVs of the disclosure to the subject. The specific route will depend upon certain variables such as the cancer cell, and can be determined by the skilled practitioner. Suitable methods of administering a NK ICV preparation described herein to a patient include by any route of in vivo administration that is suitable for delivering NK ICVs to a patient. The preferred routes of administration will be apparent to those of skill in the art, depending on the NK ICV's preparation's type of therapeutic molecule used, the target cell population, and the disease or condition experienced by the subject. Preferred methods of in vivo administration include, but are not limited to, intravenous administration, intertumoral administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue. In a particular embodiment, a preferred route of administration is by direct injection of the NK ICVs into the tumor or tissue surrounding the tumor. For example, when the tumor is a breast tumor, the preferred methods of administration include impregnation of a catheter, and direct injection into the tumor.

Intravenous, intraperitoneal, and intramuscular administrations can be performed using methods standard in the art. Aerosol (inhalation) delivery can also be performed using methods standard in the art (see, for example, Stribling et al., Proc. Natl. Acad. Sci. USA 189: 11277-11281, 1992, which is incorporated herein by reference in its entirety). Oral delivery can be performed by complexing an NK ICV preparation of the present invention to a carrier capable of withstanding degradation by digestive enzymes in the gut of an animal. Examples of such carriers include plastic capsules or tablets, such as those known in the art.

One method of local administration is by direct injection. Direct injection techniques are particularly useful for administering NK ICVs loaded with an anticancer agent, or biomolecules, to a cell or tissue that is accessible by surgery, and particularly, on or near the surface of the body. Administration of a composition locally within the area of a target cell refers to injecting the composition centimeters and preferably, millimeters from the target cell or tissue.

The appropriate dosage and treatment regimen for the methods of treatment described herein will vary with respect to the particular cancer being treated, the NK ICVs being delivered, and the specific condition of the subject. The skilled practitioner is to determine the amounts and frequency of administration on a case by case basis. In one embodiment, the administration is over a period of time until the desired effect (e.g., reduction in symptoms is achieved). In a certain embodiment, administration is 1, 2, 3, 4, 5, 6, or 7 times per week. In a particular embodiment, administration is over a period of 1, 2, 3, 4, 5, 6, 7, 8, 9, or 10 weeks. In another embodiment, administration is over a period of 2, 3, 4, 5, 6 or more months. In yet another embodiment, treatment is resumed following a period of remission.

The disclosure further provides methods for treating a subject with cancer, comprising administering an effective amount of NK ICVs disclosed herein, or pharmaceutically composition or preparation thereof. As shown in the experiments presented herein, the NK ICVs of disclosure exert beneficial cytotoxic effects against a variety of cancer cells that have different etiologies. For example, the NK ICVs exert cytotoxic effects against leukemia cells, as well as against human breast adenocarcinoma cells and cervical cancer cells. Thus, the NK ICVs of the disclosure have general applicability in treating cancers in general, and are not limited to certain types of cancer. Further, the NK ICVs disclosed herein can be loaded with anticancer agents, potentially providing for enhanced anticancer effects.

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more NK ICVs preparations described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific application. The label can also indicate directions for use of the contents, such as in the methods described herein.

The disclosure further provides that the methods and compositions described herein can be further defined by the following aspects (aspects 1 to 37):

1. A method to produce natural killer cell induced cellular vesicles (NK ICVs), comprising:

contacting natural killer cells with one or more sulfhydryl blocking agents to promote blebbing of natural killer cells to induce production of NK ICVs;

optionally, isolating or purifying the NK ICVs;

optionally, irradiating the NK ICVs prior to use in vivo.

2. The method of aspect 1, wherein the natural killer cells are mammalian natural killer cells, preferably human natural killer cells.

3. The method of aspect 2, wherein the human natural killer cells are immortalized human natural killer cells.

4. The method of aspect 3, wherein the immortalized human natural killer cells are selected from NK-92, NK-92MI, NKL, KYHG-1, and NKG.

5. The method of aspect 4, wherein the immortalized human natural killer cells are either NK-92 cells or NK-92MI cells.

6. The method of aspect 2, wherein the human natural killer cells are differentiated from human embryonic stem cells (hESCs) or induced pluripotent stem cells (iPSCs) from a human subject.

7. The method of aspect 6, wherein the iPSCs are T-cell peripheral blood cell (PBC)-derived iPSCs.

8. The method of aspect 7, wherein the T-cell PBC-derived iPSCs are differentiated to NK cells by:

culturing PBC-derived iPSCs with OP9 cells to form CD34+ differentiated cells; and

co-culturing CD34+ differentiated cells with OP9-DLL1 cells to form CD45+CD56+ natural killer cells.

9. The method of aspect 2, wherein the human natural killer cells are isolated from peripheral blood mononuclear cells or washed leukapheresis samples of one or more human subjects.

10. The method of aspect 9, wherein the human natural killer cells are isolated from peripheral blood mononuclear cells or washed leukapheresis samples using immunomagnetic negative selection, whereby non-natural killer cells are labeled with antibodies and magnetic particles and then removed with a magnet, leaving natural killer cells.

11. The method of any one of aspects 2 to 10, wherein the human natural killer cells have been genetically modified to express transgenes encoding antigen(s) and/or receptor(s).

12. The method of aspect 11, where the human natural killer cells were genetically modified by use a viral vector system.

13. The method of aspect 12, wherein the viral vector system is a lentiviral, or a retroviral vector system.

14. The method of aspect 11, wherein the human natural killer cells have been genetically modified to express a chimeric antigen receptor (CAR), preferably by use of viral vector system, more preferably by use of a lentiviral system, and wherein the NK ICVs produced are CAR-NK ICVs.

15. The method of any one of aspects 1 to 14, wherein the natural killer cells are contacted with the one or more sulfhydryl blocking agents for 3 min, 5 min, 10 min, 15 min, 20 min, 30 min, 40 min, 50 min, 1 h, 1.5 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, or a range that includes any two of the foregoing timepoints, including from 3 min to 24 h, and from 2 h to 6 h.

16. The method of aspect 15, wherein the one or more sulfhydryl blocking agents are selected from the group consisting of mercury chloride, p-chloromercuribenzene sulfonic acid, auric chloride, p-chloromercuribenzoate, chlormerodrin, meralluride sodium, iodoacetmide, paraformaldehyde, dithiothreitol, and N-ethylmaleimide.

17. The method of aspect 16, wherein the one or more sulfhydryl blocking agents are paraformaldehyde, or paraformaldehyde and dithiothreitol.

18. The method of aspect 17, wherein paraformaldehyde is used at a concentration of 20 mM, 25 mM, 30 mM, 35 mM, 40 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, or a range that includes any two of the foregoing concentrations, including from 20 mM and 250 mM, and from 25 mM to 50 mM.

19. The method of aspect 17 or aspect 18, wherein dithiothreitol is used at a concentration of 1 mM, 1.1 mM, 1.2 mM, 1.3 mM, 1.4 mM, 1.45 mM, 1.5 mM, 1.55 mM, 1.6 mM, 1.65 mM, 1.7 mM, 1.75 mM, 1.8 mM, 1.85 mM, 1.9 mM, 1.95 mM, 2.0 mM, 2.1 mM, 2.2 mM, 2.3 mM, 2.4 mM, 2.45 mM, 2.5 mM, 2.55 mM, 2.6 mM, 2.65 mM, 2.7 mM, 2.75 mM, 2.8 mM, 2.85 mM, 2.9 mM, 2.95 mM, 3.0 mM, 3.1 mM, 3.2 mM, 3.3 mM, 3.4 mM, 3.45 mM, 3.5 mM, 3.55 mM, 3.6 mM, 3.65 mM, 3.7 mM, 3.75 mM, 3.8 mM, 3.85 mM, 3.9 mM, 3.95 mM, 4 mM, or a range that includes any two of the foregoing concentrations, including from 1.0 mM to 3 mM, and 1.5 mM to 2.5 mM.

20. The method of aspect 16, wherein the one or more sulfhydryl blocking agents is N-ethylmaleimide.

21. The method of aspect 20, wherein N-ethylmaleimide is used at a concentration of 1.0 mM, 1.5 mM, 2.0 mM, 2.5 mM, 3.0 mM, 3.5 mM, 4.0 mM, 4.5 mM, 5.0 mM, 5.5 mM, 6.0 mM, 6.5 mM, 7.0 mM, 7.5 mM, 8.0 mM, 8.5 mM, 9.0 mM, 9.5 mM, 10.0 mM, 10.5 mM, 11.0 mM, 11.5 mM, 12 mM, 12.5 mM, 13.0 mM, 13.5 mM, 14.0 mM, 14.5 mM, 15.0 mM, 15.5 mM, 16.0 mM, 16.5 mM, 17.0 mM, 17.5 mM, 18.0 mM, 18.5 mM, 19.0 mM, 19.5 mM, 20.0 mM or a range that includes any two of the foregoing concentrations, including from 2.0 mM to 20.0 mM, and 2.0 mM to 5.0 mM.

22. The method of any one of aspects 1 to 21, wherein micrometer sized NK ICVs are isolated or purified.

23. The method of any one of aspects 1 to 22, wherein nanometer sized NK ICVs are isolated or purified.

24. Natural killer cell induced cellular vesicles (NK ICVs) produced by the method of any one of aspects 1 to 23.

25. The NK ICVs of aspect 24, wherein the NK ICVs are loaded with one or more small molecule therapeutic compounds or agents.

26. The NK ICVs of aspect 25, wherein the NK ICVs are loaded with one or more anticancer or chemotherapeutic agents.

27. The NK ICVs of aspect 26, wherein the one or more anticancer or chemotherapeutic agents are selected from the group of doxorubicin, daunorubicin, all-trans retinoic acid, mitoxantrone, podocalyxin, paclitaxel, and any combination thereof.

28. Chimeric antigen receptor natural killer cell induced cellular vesicles (CAR-NK ICVs) produced by the method of any one of aspects 14 to 23.

29. The CAR-NK ICVs of aspect 28, wherein the CAR-NK ICVs are loaded with one or more small molecule therapeutic compounds or agents.

30. The CAR-NK ICVs of aspect 29, wherein the CAR-NK ICVs are loaded with one or more anticancer or chemotherapeutic agents.

31. The CAR-NK ICVs of aspect 30, wherein the one or more anticancer or chemotherapeutic agents are selected from the group of doxorubicin, daunorubicin, all-trans retinoic acid, mitoxantrone, podocalyxin, paclitaxel, and any combination thereof.

32. A pharmaceutical composition comprising a pharmaceutically acceptable carrier and the NK ICVs of any one of aspects 24 to 27.

33. A method of treating a subject with cancer, comprising administering a therapeutically effective amount of the pharmaceutical composition of aspect 32 to a subject in need thereof.

34. The method of aspect 33, wherein the NK ICVs are produced from natural killer cells of the subject to be treated.

35. A pharmaceutical composition comprising a pharmaceutically acceptable carrier and the CAR-NK ICVs of any one of aspects 28 to 31.

36. A method of treating a subject with cancer, comprising administering an effective amount the pharmaceutical composition of claim 35 to a subject in need thereof.

37. The method of claim 36, wherein the CAR-NK ICVs are produced from autologous natural killer cells of the subject to be treated.

The following examples are intended to illustrate but not limit the disclosure. While they are typical of those that might be used, other procedures known to those skilled in the art may alternatively be used.

### Examples

Cell Culture: NK-92, a natural killer cell line established from human non-Hodgkin's lymphoma, was selected for preliminary studies. The cell line is promising for immunotherapy due to its ability to target and induce apoptosis in a wide variety of cancer cell types. Compared to other human NK cell lines, NK-92 cells are cytotoxic towards cancer cells even at low effector:target (E:T) ratios. NK-92 cells are obtained from American Type Culture Collection and are cultured in suspension in Alpha Minimum Essential medium without ribonucleosides and deoxyribonucleosides but with 2 mM L-glutamine and 1.5 g/L sodium bicarbonate. The cells tend to grow in aggregates that may lose viability when they are dispersed.

Protocol for inducing cellular vesicles from NK-92 cells. NK92 cells were washed three times with 1×DPBS (10 mL). After which, the NK92 cells were treated at 37° C. and 5% CO₂ in 1×DPBS with a chemical agent selected from: (a) 25 mM paraformaldehyde (PFA), (b) 25 mM PFA with 2 mM dithiothreitol (DTT), or (c) 2 mM N-ethylmaleimide (NEM), to induce blebbing for varying amounts of time.

Protocol for nano- and microscale induced cellular vesicle isolation. Cells and debris were removed by centrifugation at 1200 rpm for 5.5 min, repeated three times, each time collecting supernatant into a clean tube. Then, mICVs were isolated from the supernatant by centrifugation at 16,000×g for 10 min. After the first spin, supernatant was collected for nICV isolation while micro ICV pellets were washed with 1×DPBS. This was repeated two additional times, discarding supernatant and washing the mICV pellet with 1×DPBS. nICVs were isolated using 100 kDa Amicon® ultrafiltration at 3300×g for 15 min, repeated three times with 1×DPBS washes. Final mICVs and nICVs collected were suspended in 100 uL 1×DPBS and then characterized.

MTT assay. A solution of MTT is made in 1×DPBS or 1×PBS at a concentration of 5 mg/mL; and an MTT solvent is made comprising 4 mM HCl, 0.1% NP40 in isopropanol. The various cell lines are grown under standard culture conditions. The cell culture media is changed to serum free media for the MTT assay. For adherent cells, the cell culture media is carefully aspirated from the cells, and then serum free media and the MTT solution, as described above, is added to the plate. For suspension cells, the cells are transferred to a centrifuge tube and spun down. After the culture media is carefully aspirated off the cells, the cells are taken up in serum-free media and the MTT solution and transferred to a cell culture plate. After incubating the plate at 37° C., the MTT solvent is added to the plate. The plate is wrapped in foil and agitated on an orbital shaker for 15 minutes. The percentage of viable cells is then determined.

Characterization of mIVs and nIVs. mICVs and nICVs were imaged using a bright field microscope and inverted light microscope. The sizing of produced ICVs was measured by using dynamic light scattering (DLS). Use of transmission electron microscopy are also be used to size the produced ICVs.

Time course evaluation of induced cellular vesicle production using the blebbing agents: PFA and DTT, and NEM. As shown in FIG. 1A-B, NK92 cells grew as aggregates prior to addition of the blebbing agents. Treating the NK92 cells with PFA and DTT resulted in the production of mICVs and nICVs 2 h post treatment (see FIG. 1A). Treating the NK92 cells with NEM resulted in the production of mICVs and nICVs 2 h post treatment (see FIG. 1B). mICV production was favored over nICV production for 2 h to 4 h post treatment with PFA and DTT (see FIG. 1A) and with NEM (see FIG. 1B), while nICV production was favored over mICV production 6 h to 24 h post treatment with PFA and DTT (see FIG. 1A) and with NEM (see FIG. 1B).

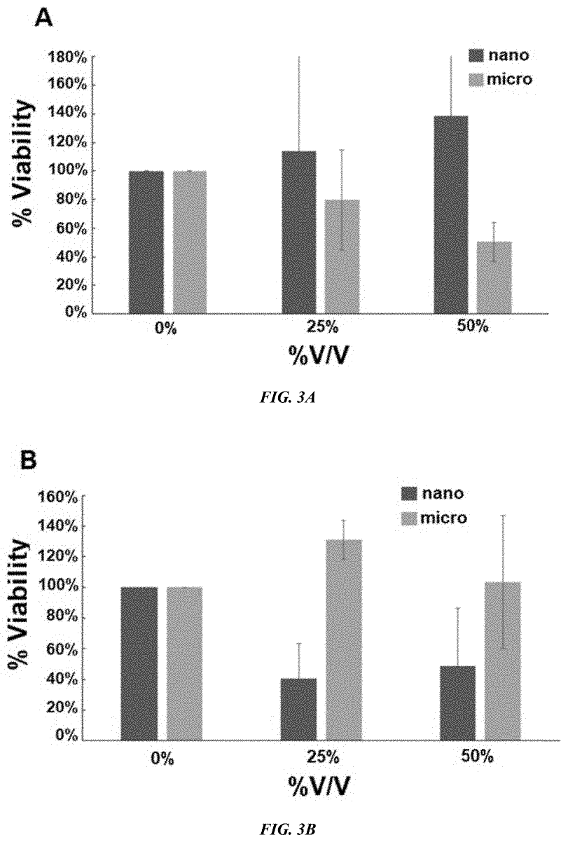

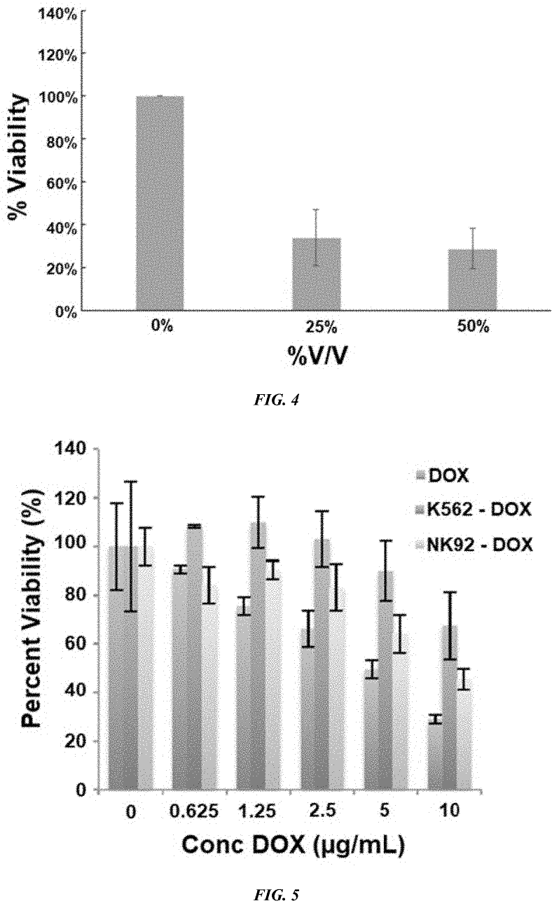

Assessing the cytotoxic effects of NK92 ICVs on various types of cancer cells. MTT assays were conducted to test the effect of NK92 ICVs on cancer cell viability. As shown in FIG. 2, NK92 nICVs produced by using the blebbing agents PFA and DTT were found to reduce the viability of K562 leukemia cancer cells in a dose dependent manner. NK92 nICVs produced by using the blebbing agent PFA or NEM also showed dose dependent toxicity with HeLa cervical cancer cells (e.g., see FIG. 3A-B). It was surprisingly found that mICVs produced by using PFA were far more cytotoxic for HeLa cells the nICVs using PFA (see FIG. 3A), while the exact opposite was found when mICVs and nICVs produced by NEM were used (see FIG. 3B). NK92 nICVs produced using PFA also demonstrated dose-dependent toxicity with MCF7 human breast adenocarcinoma cells (e.g., see FIG. 4).

Chemotherapeutics can be Loaded into NK ICVs to Enhance Toxicity. Nano-scale ICVs were produced and isolated from NK92 cells or K562 cancer cells. ICVs were then loaded with doxorubicin (DOX), a known cancer chemotherapeutic, by incubation at 37° C. DOX-loaded ICVs were tested for cytotoxicity to K562 cancer cells compared to free DOX, as assessed by using an MTT assay (see FIG. 5). Over c24 h, the tested agents exhibited the following order of K562 cytotoxicity: DOX>NK92 ICVs-DOX>K562 ICVs-DOX. The result demonstrates that while free drug may be more toxic compared to encapsulated drug at shorter time points, NK92 EB-DOX was similar and had better targeting ability than non-NK ICVs.

It will be understood that various modifications may be made without departing from the spirit and scope of this disclosure. Accordingly, other embodiments are within the scope of the following claims.

1. A method to produce natural killer cell induced cellular vesicles (NK ICVs), comprising:
contacting human natural killer cells with one or more sulfhydryl blocking agents to promote blebbing of the human natural killer cells to induce production of NK ICVs, wherein the one or more sulfhydryl blocking agents are selected from the group consisting of paraformaldehyde, and N-ethylmaleimide;
optionally, isolating or purifying the NK ICVs.

2. (canceled)

3. The method of claim 1, wherein the human natural killer cells are immortalized human natural killer cells.

4. The method of claim 3, wherein the immortalized human natural killer cells are selected from NK-92, NK-92MI, NKL, KYHG-1, and NKG.

5. (canceled)

6. The method of claim 1, wherein the human natural killer cells are differentiated from human embryonic stem cells (hESCs) or induced pluripotent stem cells (iPSCs) from a human subject.

7. The method of claim 6, wherein the iPSCs are T-cell peripheral blood cell (PBC)-derived iPSCs.

8. The method of claim 7, wherein the T-cell PBC-derived iPSCs are differentiated to NK cells by:
culturing PBC-derived iPSCs with OP9 cells to form CD34+ differentiated cells; and
co-culturing CD34+ differentiated cells with OP9-DLL1 cells to form CD45+ CD56+ natural killer cells.

9. The method of claim 1, wherein the human natural killer cells are isolated from peripheral blood mononuclear cells or washed leukapheresis samples of one or more human subjects.

10. The method of claim 9, wherein the human natural killer cells are isolated from peripheral blood mononuclear cells or washed leukapheresis samples using immunomagnetic negative selection, whereby non-natural killer cells are labeled with antibodies and magnetic particles and then removed with a magnet, leaving natural killer cells.

11. The method of claim 1, wherein the human natural killer cells have been genetically modified to express transgenes encoding antigen(s) and/or receptor(s).

12. The method of claim 11, where the human natural killer cells were genetically modified by use a viral vector system.

13. (canceled)

14. The method of claim 11, wherein the human natural killer cells have been genetically modified to express a chimeric antigen receptor (CAR), and wherein the NK ICVs produced are CAR-NK ICVs.

15-20. (canceled)

21. The method of claim 1, wherein N-ethylmaleimide is used at a concentration of 2 mM to 20 mM.

22. The method of claim 1, wherein micrometer sized NK ICVs or nanometer sized NK ICVs are isolated or purified.

23. (canceled)

24. Natural killer cell induced cellular vesicles (NK ICVs) produced by the method of claim 1.

25. (canceled)

26. The NK ICVs of claim 24, wherein the NK ICVs are loaded with one or more anticancer or chemotherapeutic agents.

27. (canceled)

28. Chimeric antigen receptor natural killer cell induced cellular vesicles (CAR-NK ICVs) produced by the method of claim 14.

29-32. (canceled)

33. A method of treating a subject with cancer, comprising administering a therapeutically effective amount of a pharmaceutical composition comprising the NK ICVs of claim 26 to a subject in need thereof.

34. The method of claim 33, wherein the NK ICVs are produced from natural killer cells of the subject to be treated.

35. (canceled)

36. A method of treating a subject with cancer, comprising administering an effective amount of a pharmaceutical composition comprising the CAR NK ICVs of claim 28 to a subject in need thereof.

37. The method of claim 36, wherein the CAR-NK ICVs are produced from autologous natural killer cells of the subject to be treated.
